(19) United States
(12) Patent Application Publication
(10) Pub. No.: US 2008/0108643 A1
(43) Pub. Date: May 8, 2008

(54) METHOD FOR TREATING AUTISM

(75) Inventor: Michael Gene Chez, Granite Bay, CA (US)

Correspondence Address: Forest Laboratories, Inc.;Attn: Charles S. Ryan, 909 3rd Avenue, New York, NY, 10022, US

(73) Assignee: FOREST LABORATORIES HOLDINGS LIMITED, Hamilton (BM)

(21) Appl. No.: 11/934,106

(22) Filed: Nov. 2, 2007

Related U.S. Application Data
(60) Provisional application No. 60/856,704, filed on Nov. 3, 2006.

(51) Int. Cl. A61K 31/439; A61P 25/00

(52) U.S. Cl. 514/289

(57) ABSTRACT

A method for treating autism comprising the step of administering an effective amount of Memantine and dextromethorphan or pharmaceutically acceptable derivatives and/or salts thereof.

This application claims the benefit of U.S. Provisional Application Ser. No. 60/856,704, filed Nov. 3, 2006, the entire disclosure of which is hereby incorporated by reference.

### FIELD OF THE INVENTION

The present invention relates in general to a method for treating autism and, more particularly, to a method for treating autism via administering effective amounts of memantine and dextromethorphan or pharmaceutically acceptable derivatives and/or salts thereof.

### BACKGROUND OF THE INVENTION

Autism is a complex developmental disability that interferes with, among other things, the normal development of the brain in the areas of social interaction and communication skills. It typically appears during the first three years of life and is the result of a neurological disorder which affects the functioning of the brain. Typically, autistic children and adults have difficulties in verbal and non-verbal communication, social interactions, and leisure or play activities.

According to the Autism Society of America (hereinafter the “ASA”), autism is generally characterized as one of five disorders coming under the umbrella of Pervasive Developmental Disorders (PDD), a category of neurological disorders characterized by severe and pervasive impairment in several areas of development, including social interaction and communications skills (DSM-IV-TR). The five disorders under PDD are Autistic Disorder, Asperger's Disorder, Childhood Disintegrative Disorder (CDD), Rett's Disorder, and PDD-Not Otherwise Specified (PDD-NOS). Specific diagnostic criteria for each of these disorders can be found in the Diagnostic & Statistical Manual of Mental Disorders (DSM-IV-TR) as distributed by the American Psychiatric Association (APA).

The most common of the Pervasive Developmental Disorders, autism affects an estimated 1 in approximately 200 births. Indeed, as of 2003-2004, as many as 1.5 million Americans are believed to have some form of autism. Such a number is on the rise inasmuch as, based on statistics from the U.S. Department of Education and other governmental agencies, autism is growing at a rate of 10-17 percent per year. At these rates, the ASA estimates that the prevalence of autism could easily reach 4 million Americans in the next decade.

The overall incidence of autism is, for the most part, globally consistent. Indeed, autism knows no racial, ethnic, or social boundaries, and family income, lifestyle, and educational levels do not affect the chance of autism's occurrence. However, it has been found to be four times more prevalent in boys than girls.

Since being first described by Dr. Leo Kanner in 1943, the understanding of autism has grown tremendously. However, the general public, and even many professionals in the medical, educational, and vocational fields, remain unaware of the effects of the disability and how to most effectively work with individuals having the disability. For example, autistic individuals may exhibit both positive and negative responses to their environment. Though some may find it surprising, many children and adults with autism may make eye contact, show affection, smile and laugh, and demonstrate a variety of other emotions, although in varying degrees.

Although autism is defined by a certain set of behaviors, it is a spectrum disorder in that its symptoms and characteristics can be present in a wide variety of combinations, from mild to severe. Therefore, autistic children and adults can exhibit any combination of the behaviors in any degree of severity. Two individuals, both with the same diagnosis, may have varying skills and display very different actions.

Indeed, every person with autism is an individual, and like all individuals, each has a unique personality and combination of characteristics. Those only mildly affected may exhibit slight delays in language or communication and may face greater challenges in social interactions. For example, one may have difficulty initiating and/or maintaining a conversation. Communication by autistic children or adults is often displayed as talking at others (for example, a monologue on a favorite subject that continues despite attempts by others to interject comments).

Autism requires those affected by it to process and respond to information in unique ways. At times, aggressive and/or self-injuries behavior may exist. The following traits, as identified by the ASA, may also be present in person with autism: Insistence on sameness or resistance to change; Difficulty in expressing needs; (i.e., uses gestures or pointing instead of words); Repeating words or phrases in place of normal, responsive language; Laughing, crying, showing distress for reasons not apparent to others; Prefers to be alone or aloof manner; Tantrum; Difficulty in mixing with others; May not want to cuddle or be cuddled; Little or no eye contact; Unresponsive to normal teaching methods; Sustained odd play; Spins objects; Inappropriate attachments to objects; Apparent over-sensitivity or under-sensitivity to pain; No real fears of danger; Noticeable physical over-activity or extreme under-activity; Uneven gross/fine motor skills; and/or Not responsive to verbal cues (i.e. acts as if deaf although hearing tests in normal range).

For most people, our senses help us to understand what we are experiencing. For example, our senses of touch, smell, sound and taste collaborate to give us a full experience of eating a ripe apple: the feel of the smooth skin as we pick it up, its sweet smell as we move it to our mouth, the crunch of the fruit being bitten into, and the juices running down our face as we enjoy the bite. For individuals with autism, however, sensory integration problems are common. In particular, their senses may be either over- or under-active. The fuzz of a kiwi may actually be experienced as painful; a sweet, fruity smell may cause a gagging reflex. Some children or adults with autism are particularly sensitive to sound, so that even the most ordinary daily noises are painful. Many professionals feel that some of the typical autism behaviors are actually a result of sensory integration difficulties.

Although there is no single known case for autism, it is generally accepted that it is caused by abnormalities in brain structure or function. The shape and structure of the brain in autistic versus non-autistic children show differences when brain scans are viewed. Currently the link between heredity, genetics and medical problems are being investigated by researchers, as well as a number of other theories. The theory of a genetic basis of the disorder is supported by the fact that, in many families, there appears to be a pattern of autism or related disabilities. While no one gene has been identified as causing autism, researchers are searching for irregular segments of genetic code that autistic children may have inherited. While researchers have not yet identified a single “trigger” that causes autism to develop, it also appears that some children are born with a susceptibility to autism.

Other researchers are investigating the possibility that under certain conditions, a cluster of unstable genes may interfere with brain development resulting in autism. Still other researchers are investigating problems during pregnancy or delivery as well as environmental factors such as viral infections, metabolic imbalances, and exposure to environmental chemicals.

According to the ASA, autism tends to occur more frequently than expected among individuals who have certain medical conditions, including Fragile X syndrome, tuberous sclerosis, congenital rubella syndrome, and untreated phenylketonuria (PKU). Some harmful substance ingested during pregnancy also have been associated with an increased risk of autism. Early in 2002, The Agency for Toxic Substances and Disease Registry (ATSDR) prepared a literature review of hazardous chemical exposures and autism and found no compelling evidence for an association; however, there was very limited research and more needs to be done.

Whatever the cause, parents can rest assured that autism is not caused by bad parenting. Children with autism and PDD are either born with the disorder or with the potential to develop it. No known psychological factors in the development of the child have been shown to cause autism. Furthermore, autism is not a mental illness; autistic children are not unruly kids who choose not to behave.

Notwithstanding the foregoing, and to the best of Applicant's knowledge, there is no cure for autism. There are however, a number of medications, developed for other conditions, which have been found to be somewhat helpful in treating a limited number of the symptoms and behaviors frequently found in individuals with autism, such as hyperactivity, impulsivity, attention difficulties, and anxiety. Examples of medications used to treat symptoms associated with autism include: Serotonin re-uptake inhibitors (e.g. clomipramine (Anafranil), fluvoxamine (Luvox) and fluoxetine (Prozac)) which have been effective in treating depression, obsessive-compulsive behaviors, and anxiety that are sometimes present in autism. Studies have shown that they may reduce the frequency and intensity of repetitive behaviors, and may decrease irritability, tantrums and aggressive behavior. Some children have shown improvements in eye contact and responsiveness. Other drugs, such as Elavil, Wellbutrin, Valium, Ativan and Xanax, require more studies to be done but may have a role in reducing behavioral symptoms.

Over the past 35 years, the most widely studied psychopharmologic agents in autism have been anti-psychotic medications. Originally developed for treating schizophrenia, these drugs have been found to decrease hyperactivity, stereotypic behaviors, withdrawal and aggression in autistic children. Four that have been approved by the FDA are clozapine (Clorazil), risperidone (Risperdal), olanzapine (Zyprexa) and quetiapine (Seroquel). However, only risperidone has been investigated in a controlled study of adults with autism. Unfortunately, like the antidepressants, these drugs all have adverse side effects, including, but not limited to, sedation.

Stimulants, such as Ritalin, Adderall, and Dexedine, used to treat hyperactivity in children with ADHD have also been prescribed for children with autism. Although a few studies have been done, they may increase focus, and decrease impulsivity and hyperactivity in autism, particularly in higher-functioning children. Unfortunately, adverse behavioral side effects are often observed.

While many of the above-identified medications do appear to be somewhat helpful in treating a limited number of the symptoms and behaviors frequently found in individuals with autism, a wide variety of side effects are associated with such medications.

It has now been surprisingly discovered that administering effective amounts of memantine and dextromethorphan or pharmaceutically acceptable salts thereof appears to substantially improve, in an apparent synergistic manner, frontal executive functions associated with autistic symptoms, including, but not limited to, speech expression and decreased preservation—among others. Furthermore, administering such medications or pharmaceutically acceptable salts thereof has not been shown to cause side effects associated with medications previously used to treat the symptoms of autism.

It is therefore an object of the present invention, to provide a method for treating autism via administering effective amounts of memantine and dextromethorphan or pharmaceutically acceptable derivatives and/or salts thereof.

### SUMMARY OF INVENTION

The present invention is directed to a method for treating autism comprising the step of administering effective amounts of a first medicament (e.g., memantine) and a second medicament (e.g., dextromethorphan) or pharmaceutically acceptable derivatives and/or salts thereof.

In accordance with the present invention, the step of administering an effective amount of a first medicament includes the step of administering an effective amount of a medicament represented by the following chemical structure: 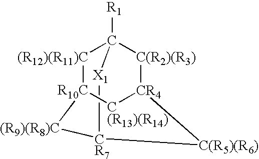
wherein X₁ comprises CH₂ or R₁₅; wherein R₁₋₁₅ are the same or different and comprise H, an amino group, a primary amine, a secondary amine, a tertiary amine, a quaternary ammonium group, a hydroxy group, a straight or branched alkyl, cycloalkyl, polycycloalkyl, heterocycloalkyl, aryl, alkaryl, aralkyl, alkoxy, alkenyl, alkynyl group containing approximately 1 to approximately 50 carbon atom(s), a silyl or siloxyl group containing approximately 1 to approximately 50 silicon atom(s), and combinations thereof.

Preferably, the step of administering an effective amount of a first medicament includes the step of administering an effective amount of a medicament represented by the following chemical structure: 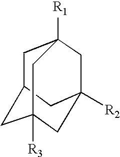
wherein R₁₋₃ are the same or different and comprise H, an amino group, a primary amine, a secondary amine, a tertiary amine, a quaternary ammonium group, a hydroxy group, a straight or branched alkyl, cycloalkyl, polycycloalkyl, heterocycloalkyl, aryl, alkaryl, aralkyl, alkoxy, alkenyl, alkynyl group containing approximately 1 to approximately 50 carbon atom(s), a silyl or siloxyl group containing approximately 1 to approximately 50 silicon atom(s), and combination thereof.

In yet another preferred embodiment of the present invention, the step of administering an effective amount of a first medicament includes the step of administering an effective amount of a medicament represented by the following chemical structure:

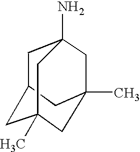

In accordance with the present invention, the step of administering an effective amount of a first medicament may include the step of administering an effective amount of a medicament represented by the following chemical structure:

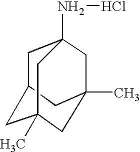

In another preferred embodiment of the present invention, the step of administering an effective amount of a first medicament includes the step of administering an effective amount of 1-amino-3,5-dimethyladamantane hydrochloride (memantine) and pharmaceutically acceptable derivatives thereof.

Preferably, the step of administering an effective amount of medicament includes the step of administering the medicament in a dosage ranging from approximately 1 mg to approximately 100 mg per day.

In yet another preferred embodiment of the present invention, the step of administering an effective amount of first medicament includes the stop of administering the medicament in a dosage ranging from approximately 5 mg to approximately 20 mg per day.

In accordance with the present invention, the step of administering an effective amount of a second medicament includes the step of administering an effective amount of a medicament represented by the following chemical structure:

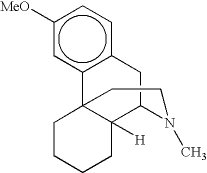

In another embodiment of the present invention, the step of administering an effective amount of a second medicament includes the step of administering an effective amount of D-(+)-3-methoxy-17-methyl-(9α,13α,14α)-morphinan and pharmaceutically acceptable salts and derivatives thereof.

Preferably, the step of administering an effective amount of a second medicament includes the step of administering the medicament in a dosage ranging from approximately 5 mg to approximately 500 mg per day.

In yet another preferred embodiment of the present invention, the step of administering an effective amount of second medicament includes the step of administering the medicament in a dosage ranging from approximately 15 mg to approximately 30 mg b.i.d.

### DETAILED DESCRIPTION OF THE INVENTION

While this invention is susceptible of embodiment in many different forms, there will herein be described in detail several specific embodiments with the understanding that the present disclosure is to be considered as an exemplification of the principles of the invention and is not intended to limit the invention to the embodiments illustrated.

In accordance with the present invention, a method for treating autism is disclosed which comprises the step of administering an effective amount of a first medicament characterized as a moderate NMDA-receptor antagonist or a pharmaceutically acceptable salt thereof, such as memantine and a second medicament, characterized as a weaker NMDA-receptor antagonist relative to the first medicament, which also acts on sigma 1 inhibition of glutamate release, such as dextromethorphan. Preferably, the NMDA-receptor antagonist of the first medicament is a moderate affinity NMDA-receptor antagonist. It will be understood that regardless of its ordinary meaning the term “moderate” will be defined in accordance with the comprehensive teachings as disclosed by Merz in its Brief Profile of Memantine available from the internet at htt://www.memantine.com/en/brief_profile/, which is hereby incorporated herein by reference in its entirety.

In one embodiment of the present invention, the first medicament is represented by the following chemical structure: 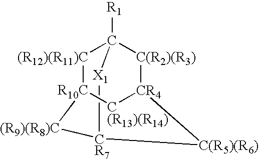
wherein X₁ comprises CH₂ or R₁₅; wherein R₁₋₁₅ are the same or different and comprise H, an amino group, a primary amine, a secondary amine, a tertiary amine, a quaternary ammonium group, a hydroxy group, a straight or branched alkyl, cycloalkyl, polocycloalkyl, heterocycloalkyl, aryl, alkaryl, aralkyl, alkoxy, alkenyl, alkynyl group containing approximately 1 to approximately 50 carbon atom(s), a silyl or siloxyl group containing approximately 1 to approximately 50 silicon atom(s), and combinations thereof.

In a second embodiment of the present invention, the first medicament is represented by the following chemical structure: 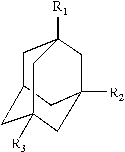
wherein R₁₋₃ are the same or different and comprise H, an amino group, a primary amine, a secondary amine, a tertiary amine, a quaternary ammonium group, a hydroxy group, a straight or branched alkyl, cycloalkyl, polocycloalkyl, heterocycloalkyl, aryl, alkaryl, aralkyl, alkoxy, alkenyl, alkynyl group containing approximately 1 to approximately 50 carbon atom(s), a silyl or siloxyl group containing approximately 1 to approximately 50 silicon atom(s), and combinations thereof.

In a third embodiment of the present invention, the first medicament is represented by the following chemical structure: 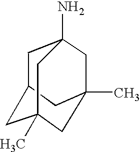
and may, specifically, comprise the hydrochloride salt provided herein below represented by the following chemical structure:

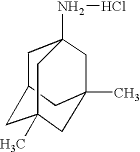

For purposes of clarity, and in an attempt to eliminate any potential ambiguity associated with the nomenclature of the above-identified medicament, it will be understood that the specific medicament provided herein above is defined as 1-amino-3,5-dimethyladamantane hydrochloride, which is commercially available from Merz under the trade name memantine.

It will be understood that an “effective amount” of one or more above-identified medicament(s) can be administered, via any one of a number of conventional means, to an autistic patient/subject. Preferably, the effective does of the first medicament ranges from approximately 1 milligram (mg) to approximately 100 mg per day, and more preferably ranges from approximately 5 mg to approximately 20 mg per day. However, the effective amount will vary depending upon the weight of the patient/subject. Preferably, the effective does of the second medicament ranges from approximately 5 milligram (mg) to approximately 200 mg per day, and more preferably ranges from approximately 15 mg to approximately 30 mg b.i.d. However, the effective amount will vary depending upon the weight of the patient/subject. Suggest administration includes: (Memantine) 2.5 mg-100 mg per day/(dextromethorphan) 5-200 mg per day. Ideally a liquid or tablet or pill containing doses of 5 mg memantine and 15 mg dextromethorphan or liquid with equivalent of 10 mg memantine with 30 mg dextromethorphan per 5 ml is possible based on current pharmacology. Ideal doses clinically based on my experience would be 5-20 mg memantine dosed divided twice daily with dextromethorphan 15-30 mg twice daily. This could be increased for either compound to range of 2.5-40 mg memantine or 10-120 mg dextromethorphan per day, or more, depending on patient tolerance and needs.

Without being bound to any particular theory, it is believed that the first and second medicaments work in theory where memantine acts to increase learning and cognitive and social language, while dextromethorphan helps mood and irritability or emotional labiality that sometime gets even worse while Memantine works cognitively.

The foregoing description merely explains and illustrates the invention and the invention is not limited thereto except insofar as the appended claims are so limited, as those skilled in the art who have the disclosure before them will be able to make modifications without departing the scope of the invention.

1. A method of treating autism comprising administering to a patient in need thereof an effective amount of dextromethorphan, or a pharmaceutically acceptable salt thereof, and a medicament represented by the following chemical structure, or a pharmaceutically acceptable salt thereof: 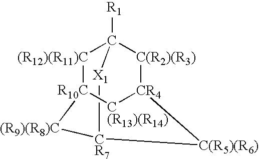
wherein X₁ comprises CH₂ or R₁₅; wherein R₁₋₁₅ are the same or different and comprise H, an amino group, a primary amine, a secondary amine, a tertiary amine, a quaternary ammonium group, a hydroxy group, a straight or branched alkyl, cycloalkyl, polycycloalkyl, heterocycloalkyl, aryl, alkaryl, aralkyl, alkoxy, alkenyl, alkynyl group containing approximately 1 to approximately 50 carbon atom(s), a silyl or siloxyl group containing approximately 1 to approximately 50 silicon atom(s), and combinations thereof.

2. The method according to claim 1, wherein the medicament is represented by the following chemical structure: 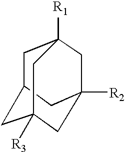
wherein R₁₋₃ are the same or different and comprise H, an amino group, a primary amine, a secondary amine, a tertiary amine, a quaternary ammonium group, a hydroxy group, a straight or branched alkyl, cycloalkyl, polycycloalkyl, heterocycloalkyl, aryl, alkaryl, aralkyl, alkoxy, alkenyl, alkynyl group containing approximately 1 to approximately 50 carbon atom(s), a silyl or siloxyl group containing approximately 1 to approximately 50 silicon atom(s), and combinations thereof.

3. The method according to claim 1, wherein the medicament is represented by the following chemical structure:

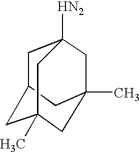

4. The method according to claim 1, wherein the medicament is represented by the following chemical structure:

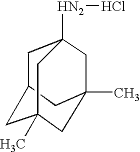

5. The method according to claim 1, wherein the dextromethorphan is administered in a dosage ranging from approximately 5 mg to approximately 200 mg per day and the medicament is administered in a dosage ranging from approximately 1 mg to approximately 100 mg per day.

6. The method according to claim 1, wherein the dextromethorphan is administered in a dosage ranging from approximately 10 mg to approximately 120 mg per day and the medicament is administered in a dosage ranging from approximately 2.5 mg to approximately 40 mg per day.

7. The method according to claim 1, wherein the dextromethorphan is administered in a dosage ranging from approximately 15 mg to approximately 30 mg per day and the medicament is administered in a dosage ranging from approximately 5 mg to approximately 20 mg per day.

8. The method according to any claim 1, wherein the dextromethorphan is administered in a dosage of 15 mg per day and the medicament is administered in a dosage of 20 mg per day
